(19) United States
(12) Patent Application Publication
(10) Pub. No.: US 2014/0182100 A1
(43) Pub. Date: Jul. 3, 2014

(54) Pre-Polarized Film Flex Circuit Based Ultrasonic Transducer

(71) Applicant: Volcano Corporation, San Diego, CA (US)

(72) Inventor: Michael Reiter, San Diego, CA (US)

(73) Assignee: Volcano Corporation, San Diego, CA (US)

(21) Appl. No.: 14/145,029

(22) Filed: Dec. 31, 2013

Related U.S. Application Data
(60) Provisional application No. 61/747,915, filed on Dec. 31, 2012.

(51) Int. Cl. H01L 41/09; A61B 8/00

(52) U.S. Cl. CPC H01L 41/09; A61B 8/4483
USPC 29/25.35

(57) ABSTRACT

The present disclosure involves a method and apparatus for making a compact and flexible ultrasonic transducer assembly that works by using existing flex circuit fabrication processes, modified flex circuit fabrication processes, and pre-polled metalized piezoelectric film, and an ASIC to create a sub 1 mm ultrasonic transducer hybrid assembly.

### PRIORITY DATA

This application claims priority to Provisional Patent Application No. 61/747,915, filed Dec. 31, 2012, and entitled “Pre-Polarized Film Flex Circuit Based Ultrasonic Transducer,” the disclosure of which is hereby incorporated by reference in its entirety.

### TECHNICAL FIELD

The present disclosure relates generally to intravascular ultrasound (IVUS) imaging, and in particular, to a method and apparatus for fabricating a compact and flexible transducer assembly used for IVUS imaging.

### BACKGROUND

Intravascular ultrasound (IVUS) imaging is widely used in interventional cardiology as a diagnostic tool for assessing a vessel, such as an artery, within the human body to determine the need for treatment, to guide intervention, and/or to assess its effectiveness. An IVUS imaging system uses ultrasound echoes to form a cross-sectional image of the vessel of interest. Typically, IVUS imaging uses a transducer on an IVUS catheter that both emits ultrasound signals (waves) and receives the reflected ultrasound signals. The emitted ultrasound signals (often referred to as ultrasound pulses) pass easily through most tissues and blood, but they are partially reflected by discontinuities arising from tissue structures (such as the various layers of the vessel wall), red blood cells, and other features of interest. The IVUS imaging system, which is connected to the IVUS catheter by way of a patient interface module, processes the received ultrasound signals (often referred to as ultrasound echoes) to produce a cross-sectional image of the vessel where the IVUS catheter is located.

IVUS catheters typically employ one or more transducers to transmit ultrasound signals and receive reflected ultrasound signals. However, the fabrication and/or assembly of conventional transducers may involve developing a rigid silicon wafer based solution which includes producing a polarizable polyvinylidene fluoride (PVDF) piezoelectric film with metallization on each side. Conventionally, pre-polled metalized PVDF film was made, which was attached it to a conventional circuit board. However, this process may be risky. For example, it may involve many time consuming processing steps and may incur a high failure rate.

Therefore, while conventional methods of producing and assembling transducers are generally adequate for their intended purposes, they have not been entirely satisfactory in every aspect.

### SUMMARY

The present disclosure provides a method and apparatus for attaching two electrical dies by wire bonding and then encasing the assembly in a protective casting that works by arranging two dies into a fixture conducive to wire bonding. Doped epoxy may be immediately dispensed over the assembly to form a near-net-shape protective cover, or Drive Can.

Both the foregoing general description and the following detailed description are exemplary and explanatory in nature and are intended to provide an understanding of the present disclosure without limiting the scope of the present disclosure. In that regard, additional aspects, features, and advantages of the present disclosure will become apparent to one skilled in the art from the following detailed description.

### BRIEF DESCRIPTIONS OF THE DRAWINGS

Aspects of the present disclosure are best understood from the following detailed description when read with the accompanying figures. It is emphasized that, in accordance with the standard practice in the industry, various features are not drawn to scale. In fact, the dimensions of the various features may be arbitrarily increased or reduced for clarity of discussion. In addition, the present disclosure may repeat reference numerals and/or letters in the various examples. This repetition is for the purpose of simplicity and clarity and does not in itself dictate a relationship between the various embodiments and/or configurations discussed.

FIG. 1 is a schematic illustration of an intravascular ultrasound (IVUS) imaging system according to various aspects of the present disclosure.

FIGS. 2-7 are diagrammatic perspective views and cross-sectional views of the transducer assembly and the various fixtures used in making the transducer assembly according to various aspects of the present disclosure.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. For example, the present disclosure provides an ultrasound imaging system described in terms of cardiovascular imaging, however, it is understood that such description is not intended to be limited to this application. In some embodiments, the ultrasound imaging system includes an intravascular imaging system. The imaging system is equally well suited to any application requiring imaging within a small cavity. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

There are primarily two types of catheters in common use today: solid-state and rotational. An exemplary solid-state catheter uses an array of transducers (typically 64) distributed around a circumference of the catheter and connected to an electronic multiplexer circuit. The multiplexer circuit selects transducers from the array for transmitting ultrasound signals and receiving reflected ultrasound signals. By stepping through a sequence of transmit-receive transducer pairs, the solid-state catheter can synthesize the effect of a mechanically scanned transducer element, but without moving parts. Since there is no rotating mechanical element, the transducer array can be placed in direct contact with blood and vessel tissue with minimal risk of vessel trauma, and the solid-state scanner can be wired directly to the imaging system with a simple electrical cable and a standard detachable electrical connector.

An exemplary rotational catheter includes a single transducer located at a tip of a flexible driveshaft that spins inside a sheath inserted into the vessel of interest. The transducer is typically oriented such that the ultrasound signals propagate generally perpendicular to an axis of the catheter. In the typical rotational catheter, a fluid-filled (e.g., saline-filled) sheath protects the vessel tissue from the spinning transducer and driveshaft while permitting ultrasound signals to freely propagate from the transducer into the tissue and back. As the driveshaft rotates (for example, at 30 revolutions per second), the transducer is periodically excited with a high voltage pulse to emit a short burst of ultrasound. The ultrasound signals are emitted from the transducer, through the fluid-filled sheath and sheath wall, in a direction generally perpendicular to an axis of rotation of the driveshaft. The same transducer then listens for returning ultrasound signals reflected from various tissue structures, and the imaging system assembles a two dimensional image of the vessel cross-section from a sequence of several hundred of these ultrasound pulse/echo acquisition sequences occurring during a single revolution of the transducer.

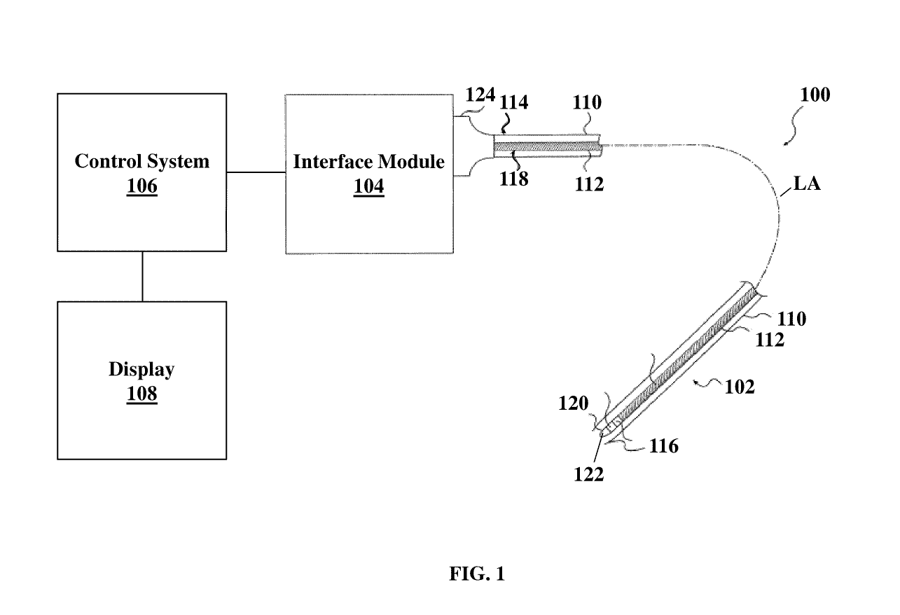

FIG. 1 is a schematic illustration of an ultrasound imaging system 100 according to various aspects of the present disclosure. In some embodiments, the ultrasound imaging system 100 includes an intravascular ultrasound imaging system (IVUS). The IVUS imaging system 100 includes an IVUS catheter 102 coupled by a patient interface module (PIM) 104 to an IVUS control system 106. The control system 106 is coupled to a monitor 108 that displays an IVUS image (such as an image generated by the IVUS system 100).

In some embodiments, the IVUS catheter 102 is a rotational IVUS catheter, which may be similar to a Revolution® Rotational IVUS Imaging Catheter available from Volcano Corporation and/or rotational IVUS catheters disclosed in U.S. Pat. No. 5,243,988 and U.S. Pat. No. 5,546,948, both of which are incorporated herein by reference in their entirety. The catheter 102 includes an elongated, flexible catheter sheath 110 (having a proximal end portion 114 and a distal end portion 116) shaped and configured for insertion into a lumen of a blood vessel (not shown). A longitudinal axis LA of the catheter 102 extends between the proximal end portion 114 and the distal end portion 116. The catheter 102 is flexible such that it can adapt to the curvature of the blood vessel during use. In that regard, the curved configuration illustrated in FIG. 1 is for exemplary purposes and in no way limits the manner in which the catheter 102 may curve in other embodiments. Generally, the catheter 102 may be configured to take on any desired straight or arcuate profile when in use.

A rotating imaging core 112 extends within the sheath 110. The imaging core 112 has a proximal end portion 118 disposed within the proximal end portion 114 of the sheath 110 and a distal end portion 120 disposed within the distal end portion 116 of the sheath 110. The distal end portion 116 of the sheath 110 and the distal end portion 120 of the imaging core 112 are inserted into the vessel of interest during operation of the IVUS imaging system 100. The usable length of the catheter 102 (for example, the portion that can be inserted into a patient, specifically the vessel of interest) can be any suitable length and can be varied depending upon the application. The proximal end portion 114 of the sheath 110 and the proximal end portion 118 of the imaging core 112 are connected to the interface module 104. The proximal end portions 114, 118 are fitted with a catheter hub 124 that is removably connected to the interface module 104. The catheter hub 124 facilitates and supports a rotational interface that provides electrical and mechanical coupling between the catheter 102 and the interface module 104.

The distal end portion 120 of the imaging core 112 includes a transducer assembly 122. The transducer assembly 122 is configured to be rotated (either by use of a motor or other rotary devices or methods) to obtain images of the vessel. The transducer assembly 122 can be of any suitable type for visualizing a vessel and, in particular, a stenosis in a vessel. In the depicted embodiment, the transducer assembly 122 includes a piezoelectric micromachined ultrasonic transducer (“PMUT”) transducer and associated circuitry, such as an application-specific integrated circuit (ASIC). An exemplary PMUT used in IVUS catheters may include a polymer piezoelectric membrane, such as that disclosed in U.S. Pat. No. 6,641,540, hereby incorporated by reference in its entirety. The PMUT transducer can provide greater than 75% bandwidth for optimum resolution in a radial direction, and a spherically-focused aperture for optimum azimuthal and elevation resolution.

The transducer assembly 122 may also include a housing having the PMUT transducer and associated circuitry disposed therein, where the housing has an opening that ultrasound signals generated by the PMUT transducer travel through. Alternatively, the transducer assembly 122 includes a capacitive micromachined ultrasonic transducer (“CMUT”). In yet another alternative embodiment, the transducer assembly 122 includes an ultrasound transducer array (for example, arrays having 16, 32, 64, or 128 elements are utilized in some embodiments).

The rotation of the imaging core 112 within the sheath 110 is controlled by the interface module 104, which provides user interface controls that can be manipulated by a user. The interface module 104 can receive, analyze, and/or display information received through the imaging core 112. It will be appreciated that any suitable functionality, controls, information processing and analysis, and display can be incorporated into the interface module 104. In an example, the interface module 104 receives data corresponding to ultrasound signals (echoes) detected by the imaging core 112 and forwards the received echo data to the control system 106. In an example, the interface module 104 performs preliminary processing of the echo data prior to transmitting the echo data to the control system 106. The interface module 104 may perform amplification, filtering, and/or aggregating of the echo data. The interface module 104 can also supply high- and low-voltage DC power to support operation of the catheter 102 including the circuitry within the transducer assembly 122.

In some embodiments, wires associated with the IVUS imaging system 100 extend from the control system 106 to the interface module 104 such that signals from the control system 106 can be communicated to the interface module 104 and/or vice versa. In some embodiments, the control system 106 communicates wirelessly with the interface module 104. Similarly, it is understood that, in some embodiments, wires associated with the IVUS imaging system 100 extend from the control system 106 to the monitor 108 such that signals from the control system 106 can be communicated to the monitor 108 and/or vice versa. In some embodiments, the control system 106 communicates wirelessly with the monitor 108.

An ultrasound transducer can be included in the IVUS imaging system 100, for example in the transducer assembly 122. The ultrasonic transducer has a small size and achieves a high resolution, so that it is well suited for intravascular imaging. In some embodiments, the ultrasonic transducer has a size on the order of tens or hundreds of microns, can operate in a frequency range between about 1 mega-Hertz (MHz) to about 135 MHz, and can provide sub 50 micron resolution while providing depth penetration of at least 10 millimeters (mm). Furthermore, the ultrasonic transducer is also shaped in a manner to allow a developer to define a target focus area based on a deflection depth of a transducer aperture, thereby generating an image that is useful for defining vessel morphology, beyond the surface characteristics. The various aspects of the ultrasound transducer and its fabrication are discussed in greater detail below.

In certain embodiments, the ultrasound transducer is a piezoelectric micromachined ultrasound transducer (PMUT). In other embodiments, the transducer may include an alternative type of transducer. Additional features can be added in the ultrasound transducer, and some of the features described below can be replaced or eliminated for additional embodiments of the ultrasound transducer. For additional details of fabricating such ultrasonic transducer, refer to U.S. Provisional Application 61/745,212, titled “Methods and Apparatus for Focusing Miniature Ultrasound Transducers” to Dylan Van Hoven, filed on Dec. 21, 2012, attorney docket 44755.1061, Provisional U.S. Patent Application 61/745,091 to Dylan Van Hoven, filed on December 21, entitled “Preparation and Application of a Piezoelectric Film for an Ultrasound Transducer”, and attorney docket 44755.1060, Provisional U.S. Patent Application No. 61/646,080 titled “DEVICE AND SYSTEM FOR IMAGING AND BLOOD FLOW VELOCITY MEASUREMENT” (Attorney Docket No. 44755.817/01-0145-US) filed on May 11, 2012, Provisional U.S. Patent Application No. 61/646,074 titled “ULTRASOUND CATHETER FOR IMAGING AND BLOOD FLOW MEASUREMENT” (Attorney Docket No. 44755.961) filed on May 11, 2012, and Provisional U.S. Patent Application No. 61/646,062 titled “Circuit Architectures and Electrical Interfaces for Rotational Intravascular Ultrasound (IVUS) Devices” (Attorney Docket No. 44755.838) filed on May 11, 2012, the contents of each of which are hereby incorporated by reference in their entirety.

Traditionally, the fabrication and/or assembly of conventional transducers may involve developing a rigid silicon wafer based solution which includes producing a polarizable polyvinylidene fluoride (PVDF) piezoelectric film with metallization on each side. Conventionally, pre-polled metalized PVDF film was made, which was attached it to a conventional circuit board. However, this process may be risky. For example, it may involve many time consuming processing steps and may incur a high failure rate. Modifying this process to use a micro-flex may be considered a less risky evolution.

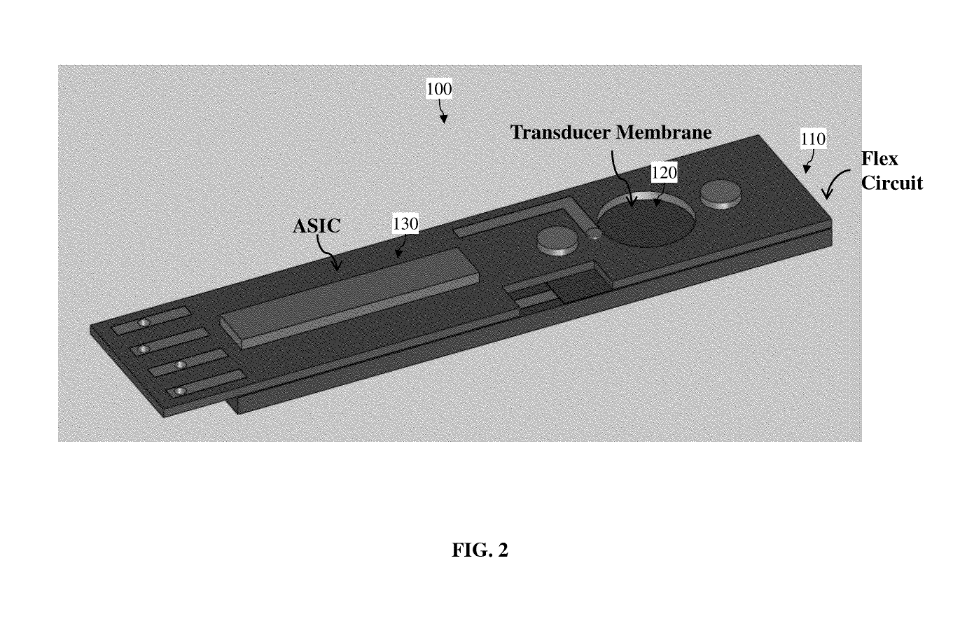

Therefore, the present disclosure involves a method and apparatus for making a compact and flexible ultrasonic transducer assembly (an embodiment of which is shown as a transducer assembly 100 in FIG. 2) that works by using existing flex circuit (an embodiment of which is shown as a flex circuit 110 of FIG. 2) fabrication processes, modified flex circuit fabrication processes, and pre-polled metalized piezoelectric film (an embodiment of which is shown as a transducer membrane 120 in FIG. 2), and an ASIC (an embodiment of which is shown as an ASIC 130 in FIG. 2) to create a sub 1 mm ultrasonic transducer hybrid assembly. By doing so, the present disclosure offers numerous advantages over the prior art. Some of these advantages include:
Pre-Polarized Film: Allows the use of an off-the-shelf polarized metalized Piezoelectric film, while providing a flexible platform for the attachment of an ASIC, and/or other electrical devices, components, wires, etc.
Doppler/Forward Looking: Flex circuit lends itself to forward looking and Doppler type imagers, for example those as described in Provisional U.S. Patent Application No. 61/646,080 titled “DEVICE AND SYSTEM FOR IMAGING AND BLOOD FLOW VELOCITY MEASUREMENT” (Attorney Docket No. 44755.817/01-0145-US) and Provisional U.S. Patent Application No. 61/646,074 titled “ULTRASOUND CATHETER FOR IMAGING AND BLOOD FLOW MEASUREMENT” (Attorney Docket No. 44755.961), and Provisional U.S. Patent Application No. 61/646,062 titled “Circuit Architectures and Electrical Interfaces for Rotational Intravascular Ultrasound (IVUS) Devices” (Attorney Docket No. 44755.838), each filed on May 11, 2012 and each of which is hereby incorporated by reference in its entirety.
Flex circuit can follow the shape of the carrier Can, thereby providing the option of parabolic shaping of the PVDF by virtue of the can.

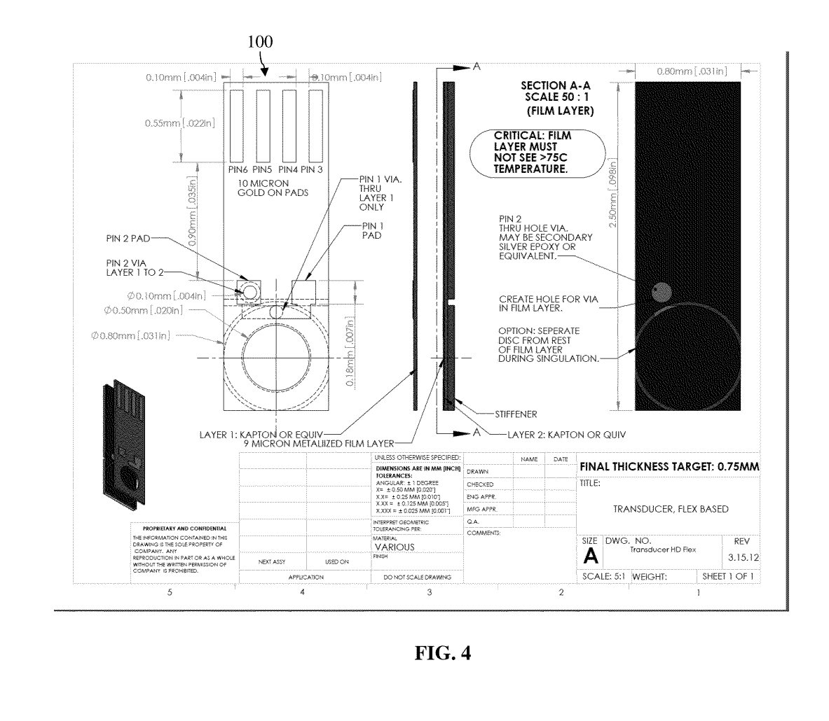

According to the various aspects of the present disclosure, a commercially available Piezoelectric film (e.g., transducer membrane 120) is formed to the desired circular shape within a flex circuit (e.g., flex circuit 110) by one of several means, and allowance for attachment of an ASIC (e.g., ASIC 130) or other components. The method steps of the present disclosure are now discussed in more detail in view of FIGS. 2-7.

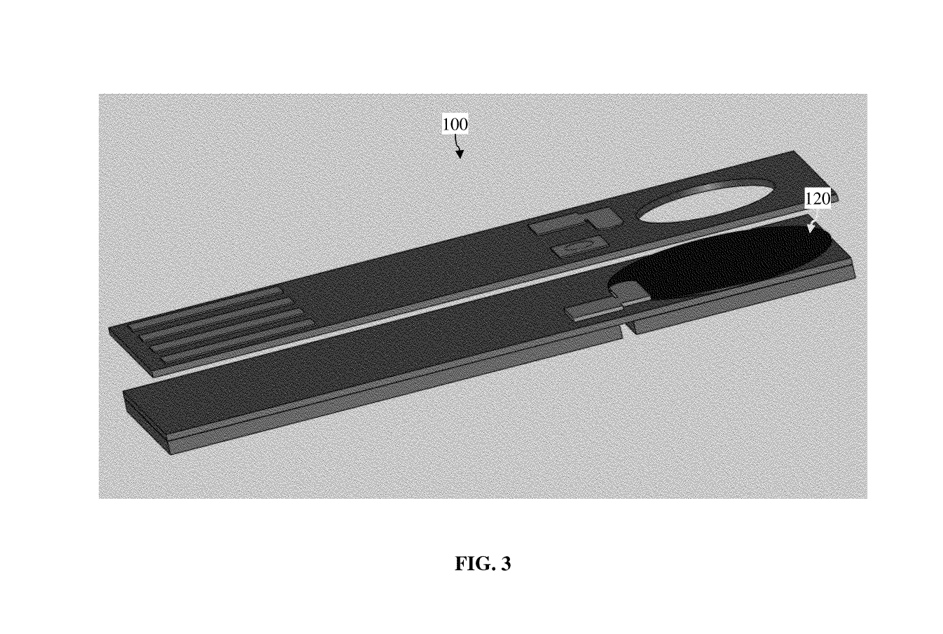

An example transducer assembly 100 according to the present disclosure is shown in FIGS. 1 and 2, which include diagrammatic perspective and exploded views of the transducer assembly 100, respectively. FIG. 3 also shows a top view sketch of the transducer assembly 100. A flex circuit 110 may be used to support and provide interconnections for a miniature ultrasonic transducer 120 and an ASIC 130. For additional details of the flex circuit, the ASIC, and the transducer (and the fabrication thereof), refer to U.S. Provisional Patent Application 61/745,091 to Dylan Van Hoven, filed on December 21, entitled “Preparation and Application of a Piezoelectric Film for an Ultrasound Transducer”, and attorney docket 44755.1060, U.S. Provisional Application 61/745,212, titled “Methods and Apparatus for Focusing Miniature Ultrasound Transducers” to Dylan Van Hoven, filed on Dec. 21, 2012, attorney docket 44755.1061, and U.S. Provisional Application 61/747,153, entitled “Transducer Assembly for an Imaging Device”, filed on Dec. 28, 2012, attorney docket 44755.1063, U.S. Provisional Application 61/747,506, titled “Layout and Method of Singulating Miniature Ultrasonic Transducers” to Cheryl D. Rice, filed on Dec. 31, 2012, attorney docket 44755.1098, and U.S. Provisional Application 61/747,498, entitled “Ultrasonic Transducer Electrode Assembly”, to Cheryl D. Rice, filed on Dec. 31, 2012, attorney docket 44755.1097, the contents of each which are incorporated by reference herein in its entirety.

Creating the Disc:

### Option 1: Pre-Cut.

Adhere a complete layer of the FILM 120 to the bottom layer Kapton (material used for the flex circuit 110) using PSA. The FILM is pre-cut with thru holes and an approximated cut out of the disc.

### Option 2: Cut in Place.

Adhere a complete layer of the FILM 120 to the bottom layer Kapton using PSA. Laser ablate just the FILM to create the thru holes and the disc.

### Option 3: Strategically Dampen.

Place a complete layer of FILM 120 to the bottom layer Kapton using PSA. Acoustically dampen all areas, except the desired disc. Could be done by doping the PSA, or adding a doped dampening layer.

Connecting to the Electrode Layers:

### Option 1: Filled Via.

A ‘filled via’ approach is used, however, due to the temperature constrains of PVDF a room temperature conductive epoxy (Ex. Silver Epoxy) or a low temperature solder (see below) is used to fill the vias and connect the top traces to the top-side pad, and bottom electrodes to the bottom-side pad. Low temperature soldering alloys (Melt<80 C).

### Option 2: Wire Bond.

A wire bonder could be used to make the same connection as the filled via material above, but would require a much larger opening (target) for the wire bond. The wire bond thereafter is quite fragile and would require a secondary potting operation to strengthen.

Re-Polling: In the event the PVDF film requires re-polarization, or an un-polarized film is desirable, then the film can be polarized by attachment of temporary electrical contacts to the pads connected to the upper and lower electrodes prior to attachment of any electrical components.

Stiffener:
FR4 or Cirlex stiffeners attached to the bottom of the flex allow pressurized deflection of the PVDF film. By segmenting the stiffener, the active film area can be angled, allowing forward looking and Dopler configurations.
Once in place, the device is rigidly potted, making any stiffener redundant. Omitting the stiffener reduces the thickness of the device, while still allowing the deflection process to be performed. In this configuration, the Can housing must be pre-filled or machined to create a platform for the device; as the device lacks any significant rigidness of its own.

ASIC and Component Attachment:
Option 1: Secondary components can be attached by the same methods described for the filled vias. An automated pick and place process (SMT) could be used for high volume assembly.
Option 2: Secondary components may be epoxied in place upside-down to the flex, and a wire bond operation used to make the electrical connections between flex circuit pads and component pads.
Option 3: Secondary components may be wire bonded in a fixture, then epoxied together.

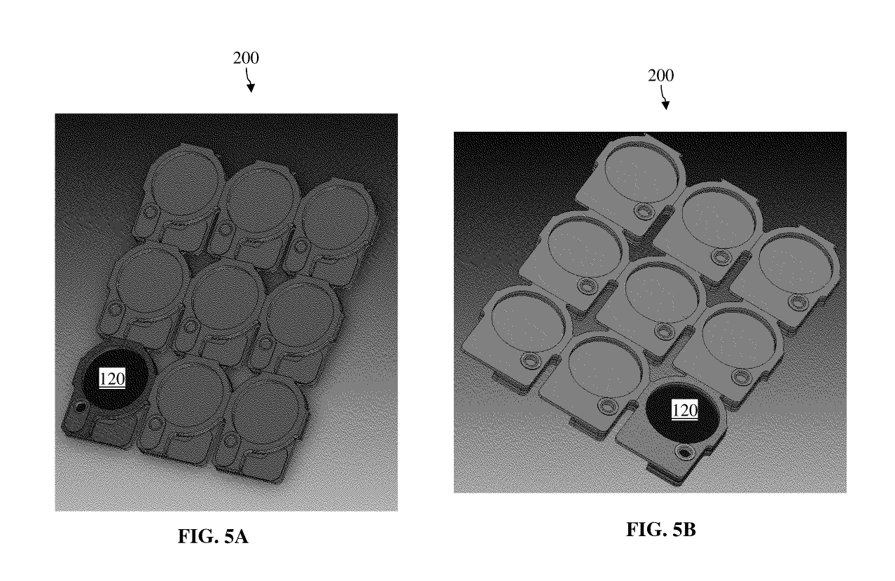

With reference to FIGS. 5A and 5B, a perspective view of a fixture 200 used to fabricate the transducer 120 is illustrated. FIGS. 5A and 5B show opposite sides of the fixture 200. Each layer of the transducer may be laser cut, water-jet cut, etched, or mechanically cut. The layers are placed in the fixture. Partial vacuum is pulled to reduce air between the layers. A mechanical press is then performed to activate the pressure sensitive adhesive (PSA). The PSA layer may be disposed between the top Kapton, the middle PVDF, and the bottom Kapton. By doing this, the PVDF material used is reduced. In addition, the activated PVDF material outside of the transducer disc area is also minimized.

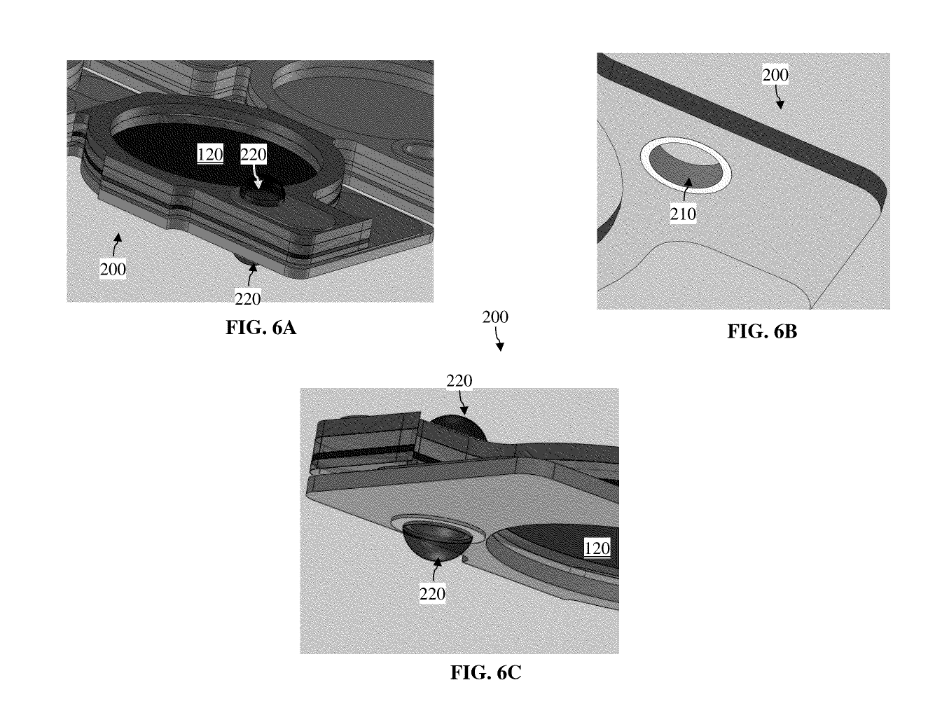

With reference to FIGS. 6A, 6B, and 6C, various fragmentary perspective views of a portion of the fixture 200 of FIGS. 5A-5B are illustrated. The top-side pad contacts the top-side PVDF 120 by a plated thru hole 210 connected to a reverse-side ring contact. To insure electrical contact (not rely on mechanical contact alone), silver epoxy 220 is dispensed into the plated thru hole. The bottom-side uses the same approach with slightly different geometry. The PVDF 120 is then backfilled and deflected, for example in a manner similar to that as described in U.S. Provisional Patent Application 61/745,344, entitled “Method and Apparatus for Shaping Transducer Membranes”, filed on Dec. 21, 2012, attorney docket 44755.1094, the content of which is hereby incorporated by reference in its entirety. It is also understood that common working-piece coupon sizes would allow interchangeability.

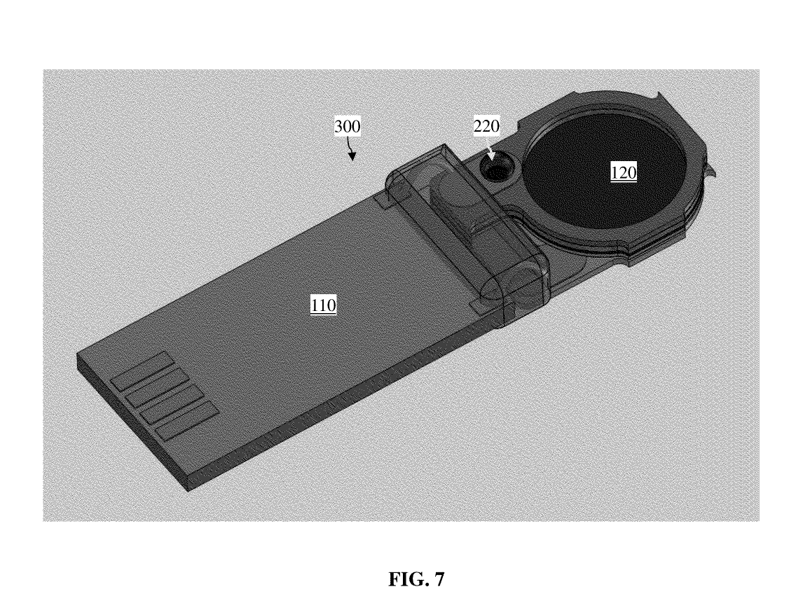

With reference to FIG. 7, an embodiment of a completed MEMS-ASIC hybrid transducer assembly 300 is illustrated according to the various aspects of the present disclosure. For additional improvement, the bottom PSA layer could be doped with acoustical dampening material, and extended underneath the PVDF disc area. The parabolic shape would then be formed during compression of the flex by ball shaped features in the compression tool which would align with each PVDF disc

Persons skilled in the art will recognize that the apparatus, systems, and methods described above can be modified in various ways. Accordingly, persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. A method of making a compact and flexible ultrasonic transducer assembly, comprising:
providing a pre-polarized piezoelectric film;
transforming the pre-polarized piezoelectric film into a circular shaped film;
placing the circular shaped film into a fixture;
providing a flex circuit;
attaching the fixture to the flex circuit; and
attaching an Application Specific Integrated Circuit (ASIC) to the flex circuit.
